(19) United States
(12) Patent Application Publication
(10) Pub. No.: US 2013/0197428 A1
(43) Pub. Date: Aug. 1, 2013

(54) Peritoneal Dialysis Method

(71) Applicant: Cosme Cruz, Chicago, IL (US)

(72) Inventor: Cosme Cruz, Chicago, IL (US)

(21) Appl. No.: 13/478,615

(22) Filed: May 23, 2012

Related U.S. Application Data
(60) Provisional application No. 61/490,324, filed on May 26, 2011.

(51) Int. Cl. A61M 1/28

(52) U.S. Cl. CPC A61M 1/287
USPC 604/28

(57) ABSTRACT

A method of dialyzing renal failure patients via the peritoneum (P.O.) using a series of innovations in the dialysate composition aiming at preventing the intra vascular or intra peritoneal formation and deposition of calcium/phosphate compounds which having no excretory pathway are deposited in soft tissue and are the cause of cardiovascular related morbidity and mortality. Additionally this method allows for the use of the physiologic buffer (bicarbonate) which will improve the acid base balance and prolong the functional life of the peritoneum as the dialysis membrane.

### FIELD OF THE INVENTION

The invention relates to methods of dialysis. More specifically the invention relates to methods of performing peritoneal dialysis using a dialysis solution devoid of divalent ions (Ca and Mg ions) which reduces the possibility of calcium overload leading to soft tissue calcification which as it involves the cardiovascular system leads to calcific cardio vascular disease, the most common cause of death in dialysis patients.

### BACKGROUND OF THE INVENTION

The kidneys have an important role in maintaining health. When healthy, the kidneys maintain the body's internal equilibrium of water and dissolved minerals (sodium. potassium, chloride, calcium, phosphorus, magnesium, etc.). Those acidic end products of metabolism that the body cannot get rid of via respiration are excreted through the kidneys. The kidneys also function as part of the endocrine system producing erythropoietin and calcitriol. Erythropoietin is involved in the production of red blood cells and calcitriol plays a role in bone remodeling.

As renal function decreases phosphate retention ensues altering the bone metabolism. Hyperphosphafemia causes calcium levels to drop and the chain of events leading to renal bone disease. As effective renal mass decreases the ability to hydroxyl ate Vit D decreases and low Vit D levels also contributes to what is known as kidney disease-mineral bone disorder (KD-MBD).

Renal failure is described as a decrease in the glomerular filtration rate. Biochemically, renal failure is typically detected by an elevated serum creatinine level. Problems frequently encountered in kidney malfunction include abnormal fluid levels in the body, deranged acid levels, abnormal levels of potassium. calcium, phosphate, and (in the longer term) anemia as well as abnormal bone remodeling. Depending on the cause, hematuria (blood loss in the urine) and proteinuria (protein loss in the urine) may occur.

The cause of renal failure can be divided into two categories: acute kidney injury or chronic kidney disease. The type of renal failure is determined by the trend in the serum creatinine. Other factors which may help differentiate acute kidney injury from chronic kidney disease include anemia and the kidney size on ultrasound. Chronic kidney disease generally leads to anemia and small kidney size.

Symptoms can vary from person to person. Someone in early-stage kidney disease may not feel sick or notice symptoms as they occur. When kidneys fail to filter properly, waste accumulates in the blood and the body, a condition called azotaemia. Very low levels of azotaemia may produce few, if any, symptoms. As the disease progresses, symptoms become noticeable (if the failure is of sufficient degree to cause symptoms). Renal failure, accompanied by noticeable symptoms, is termed uraemia.

Dialysis is an imperfect treatment to replace kidney function; it replaces some functions through diffusion (waste removal) and ultrafiltration (flUid removal), but it does not correct the endocrine functions of the kidney.

Dialysis works on the prinCiples of the diffusion of solutes and ultrafiltration of fluid across a semi-permeable membrane. Diffusion describes a property of substances in water. Substances in water tend to move from an area of high concentration to an area of low concentration. Blood flows along one side of a semi-permeable membrane, and a dialysate, or special dialysis fluid, flows along the opposite side. A semipermeable membrane is a thin layer of material that contains various-sized holes, or pores. Smaller solutes and fluid pass through the membrane, but the membrane blocks the passage of larger substances (for example, red blood cells, large proteins, etc.).

The two main types of dialysis, hemodialysis and peritoneal dialysis, remove wastes and excess water from the blood in different ways. Hemodialysis (HD) removes wastes and water by circulating blood outside the body through an external filter, called a dialyzer, that contains a semipermeable membrane. The blood flows in one direction and the dialysate flows in the opposite direction. The counter-current flow of the blood and dialysate maximizes the concentration gradient of solutes between the blood and dialysate, which helps to remove more urea and creatinine from the blood. The concentrations of solutes (for example potassium, phosphorus, and urea) are undesirably high in the blood, but low or absent in the dialysis solution. Constant replacement of the dialysate ensures that the concentration of undesired solutes is kept low on this side of the membrane. The dialysis solution has levels of minerals such as potassium and calcium that are similar to their natural concentration in healthy blood.

In peritoneal dialysis (PD), wastes and water are removed from the blood inside the body using the peritoneal membrane of the peritoneum as a natural semipermeable membrane. Wastes and excess water move from the blood, across the peritoneal membrane, and into a special dialysis solution, called dialysate, in the abdominal cavity which solution has a composition similar to the extra cellular fluids but devoid of the substances you want to remove.

In peritoneal dialysis, a sterile solution of minerals and glucose is run through a tube into the peritoneal cavity, where the peritoneal membrane acts as a semipermeable membrane. The peritoneal membrane or peritoneum is a layer of tissue containing blood vessels that lines and surrounds the peritoneal, or abdominal, cavity and the internal abdominal organs (stomach, spleen, liver, and intestines). The dialysate is left there for a period of time to absorb waste products, then it is drained out through the tube and discarded. This cycle or “exchange” is normally repeated 3-4 times during the day, (sometimes more often overnight with an automated system). An “exchange” occurs each time the dialysate fills and empties from the abdomen. Dwell time means the time the dialysate stays in the patient's abdominal cavity—wastes, chemicals and extra fluid moves from the patient's blood to the dialysate across the peritoneum.

A drain process occurs after the dwell time; the dialysate full with waste products and extra fluid is drained out of the patient's blood. Ultrafiltration occurs via osmosis; the dialysis solution used contains a high concentration of glucose, and the resulting osmotic pressure causes fluid to move from the blood into the dialysate. As a result, more fluid is drained than was instilled.

Traditional dialysis methods have been used to arrest many of the symptoms of renal failure. Dialysis solutions commonly contain sugars such as dextrose, or a dextrose polymer as well as other components to assist in maintaining blood functionality. These solutions contain electrolytes such as sodium, chloride, calcium and magnesium to maintain the functionality of the neurological system (brain and nervous system).

Examples of these dialysate solutions may be found for example in U.S. Pat. No. 6,673,376 which teaches dialysis solution of acid and carbonate which allows the use of solid acid sources to limit the emission of CO2. Wu et al., U.S. Pat. No. 6,812,222 teaches a peritoneal dialysis solution of amino sugars designed to promote reversal of water by diffusion. Naggi et al., U.S. Pat. No. 7,208,479 teaches a peritoneal dialysis solution of glucose compounds that are heat stable under sterilization conditions. Martis et al., U.S. Pat. No. 7,618,392 teaches methods and compositions for detection of microbial contaminants in peritoneal dialysis compositions. Both of U.S. Pat. Nos. 6,803,363 and 7,550,446 also teach dialysis solutions and plasma expanders.

Apart from issues of pH balance, dehydration and irregular neurological function, another common problem that may occur with kidney dialysis patients is the buildup of calcium compounds on the vessels and the viscerae. This may lead to any number of problems including vascular insufficiency and heart failure.

Currently research has yielded no effective means to eliminate calcium and/or magnesium from HD or PD fluid formulations. Heretofore, such problems have not been successfully addressed by the prior art. As such, there is a need for dialysis solutions which reduce the calcifying effects of dialysis while supporting renal function.

### SUMMARY OF THE INVENTION

These are two precedents supporting the rationale to remove calcium/magnesium from dialysis solution. Peritoneal dialysis solutions in current use are devoid of potassium. Hypokalemia (IOW potassium plasma levels) is of more known consequences and is not regulated as is hypocalcemia. In acute hypokalemic (low K+) red cell and muscle destruction (lysis) are corrective but potentially lethal. In contrast, acute hypocalcemia (low calcium) can go clinically unnoticed due to the fact that there are abundant calcium stores in the skeleton. These calcium stores can be used through the action of the parathyroid hormone to maintain the plasma calcium level necessary for the normal metabolism.

Obviously it is assumed that dialysis patients are potentially if not de facto hypercalemic. A negative Ca++ balance dialysis is desirable. If one assumes hypercalemic dialysis patient including patients on peritoneal dialysis have abnormally high total body calcium content which eventually results in their demise. Eliminating calcium from the dialysis solution makes perfect sense. Magnesium exists in such a small amount in dialysate and the potential loses of Mg from Mg free dialysis so small and easy to replace that it becomes a minor issue.

It has been shown that lowering dialysate calcium over time causes a reducing of skin Ca++ content (soft tissue) of hemodialysis patients. Zinn et al., “Reduction in dialysate calcium concentration lowers skin calcium content in patients on chronic hemodialysis”, Trans. Am. Soc. Artif. Intern Organs XXVI, 1980.

Through the action of Paraltronmone (PTH) the vast calcium stores in the skeleton can be tapped to maintain physiologic plasma levels of calcium, necessary for normal metabolism.

In accordance with a first aspect of the invention, there is provided a method of delivering a solution to a kidney dialysis patient comprising the steps of withdrawing solutions from the dialysis patient's peritoneum; and infusing a solution comprising sugar ranging from 1.5-6.0%; an effective amount of buffering agent (preferably NaHCO3); chloride; sodium; and a balance of water.

In accordance with a further aspect of the invention, there is provided a method of delivering an aqueous dialysis solution comprising the steps of withdrawing fluid from the peritoneum; and infusing the aqueous dialysis solution into the peritoneum, the aqueous solution comprising a dextrose polymer, a buffering agent (NaHC03), and sodium chloride; wherein the solution has a pH ranging from about 7.3 to 7.5, a sodium concentration of about 100 to 120 mEq/L, and a chloride concentration of about 80 to 100 mEqs/L.

Even further, there is provided a peritoneal dialysis solution comprising sodium and chloride, an amount of dextrose polymer, a carbonate buffering agent present in an amount necessary to provide a pH ranging from about 7.0 to 7.4, and a balance of water wherein the solution is free of calcium and free of magnesium.

The timing for a new treatment modality for patients suffering from End-Stage Renal Disease (ESRD) could not be better. In-Center Hemodialysis (HD), is a costly treatment modality, fraught with numerous impediments due to a highly individualized approach to its prescription and delivery. It is based on an ultra-efficient and “fast” treatment schedule of, on average, 12 hours per week (240 min. 3× per week).

Thought by many as failing to rehabilitate patients, and limiting of their lifestyle, the rapid fluid and toxin removal associated with HD creates fluctuations in body fluid and chemical composition. Severe dietary restrictions are often needed. Patient non-compliance is commonplace and results in the frequent need for hospitalizations, due to serious morbid often leading to mortality. The relatively recent awareness of yet one more serious and often fatal complication of HD makes any advances even more pertinent: the development of soft tissue (primarily cardiac and vascular) calcification. This issue has increasingly been identified as the “Achilles heel” of the ESRD replacement therapy.

As an alternative to HD, peritoneal dialysis (“PD”) offers some advantages such as being continuous, allowing the patient to adapt the dialysis schedules to their needs, having virtually no dietary restrictions, allowing the patient freedom to work and travel, and restoring reproductive function, among other benefits. Peritoneal dialysis, however, suffers from lack of technological refinements, with virtually no major advancement in the last twenty years.

Inefficient and easily contaminable equipment and delivery systems, coupled with irritating, unphysiologic dialysis solutions that shorten the effective life of the peritoneal membrane (the actual dialysis surface) have contributed to a rather limited utilization of this technique. As in HD, but not to the same degree, soft tissue calcification and slowing of the normal bone remodeling process are also recognized as significant problems in acute need of solution.

Calcium-based phosphate chelating agents (with additional magnesium sulfate and, possibly, minute amounts of zinc), may improve management of the Dialysis Related Bone Disease (R.O.D). The chelating of phosphate would take place in the intestine, and the calcium/phosphate products would be eliminated in the excrement. This scenario is more appropriate, as opposed to the present situation where the phosphate chelating is done in the blood stream or the peritoneal space by the calcium present in the dialysis fluid. This “intra vascular” chelating of the abnormally high phosphate (a universal occurrence in ESRD) is the phenomenon responsible for soft tissue calcification seen in HD and, to a lesser extent, in PD. Both leading to early death due to cardio-vascular calcification.

### BRIEF DESCRIPTION OF THE FIGURES

FIGS. 1-3 are dual energy x ray absorptometry of the patient treated in the Example.

### DETAILED DESCRIPTION OF THE PREFERRED EMBODIMENTS

In accordance with the invention, there is provided a method of delivering a solution to a kidney dialysis patient. The method includes withdrawing solutions from the dialysis patient's peritoneum; and infusing a solution comprising an amount of sugar, an amount of buffering agent; an amount of sodium and chloride; and a balance of water.

A patient's blood serum contains several components including, for example, proteins, carbohydrates, nucleic acids, and various ions. Typically, a dialysate composition prescribed by a physician is chosen to reduce, increase, or normalize the concentration of a particular component in the serum. Several cations may be prescriptively included as part of the dialysate composition. The invention is a method of dialysing a renal patient with a composition that is free of calcium, free of magnesium or free of both.

In a related embodiment, the present invention provides dry compositions (e.g., tablets, pellets, powder, etc.) which, upon mixing with water, provide a dialysate precursor composition having the electrolyte concentrations recited above. Preferably the compositions used in the method of the invention are free of calcium and its ions or magnesium and its ions, or both.

### Sugar

One function of the composition used in the method of the invention is to work as an osmotic agent. Renal failure often causes the build up of water or water based fluids. Any number of agents may be used to accomplish this function including sugars, amino acids, polymers and mixtures thereof.

In one embodiment, the dialysate composition includes one or more sugars selected from glucose (preferably dextrose), poly(glucose) (preferably, poly(dextrose), e.g., icodextrin), and fructose at a concentration of less than about 45 giL. Instead, or in addition to sugar, the dialysate composition may contain one or more amino acids. Preferably the sugar used in the composition of the invention comprises dextrose or D-glucose.

Glucose CSH120S, also known as D-glucose, dextrose (or grape sugar), is a simple sugar (monosaccharide) and an important carbohydrate in biology. Cells use it as a source of energy and a metabolic intermediate. Glucose is one of the main products of photosynthesis and starts cellular respiration.

Glucose exists in several different structures, but all of these structures can be divided into two families of mirror-images (stereoisomers). Only one set of these isomers exists in nature, those derived from the “right-handed form” of glucose, denoted D-glucose. D-glucose is often referred to as dextrose. The term dextrose is derived from dextrorotatory glucose. Solutions of dextrose rotate polarized light to the right. Starch and cellulose are polymers derived from the dehydration of D-glucose. The other stereoisomer, called L-glucose, is hardly found in nature.

In its fleeting open-chain form, the glucose molecule has an open (as opposed to cyclic) and unbranched backbone of six carbon atoms, C-1 through C-6; where C-1 is part of an aldehyde group H(C═O)—, and each of the other five carbons bears one hydroxyl group —OH. The remaining bonds of the backbone carbons are satisfied by hydrogen atoms —H. Therefore glucose is an hexose and an aldose, or an aldohexose.

Each of the four carbons C-2 through C-5 is chiral, meaning that its four bonds connect to four distinct parts of the molecule. (Carbon C-2, for example, connects to —(C═O)H, —OH, —H, and —(CHOH)4H.) In D-glucose, these four parts must be in a specific three-dimensional arrangement. Namely, when the molecule is drawn in the Fischer projection, the hydroxyls on C-2, C-4, and C-5 must be on the right side, while that on C-3 must be on the left side.

The positions of those four hydroxyls are exactly reversed in the Fischer diagram of L-Glucose. D- and L-glucose are two of the 16 possible aldohexoses; the other 14 are allose, altrose, mannose, gulose, idose, galactose, and talose, each with two isomers, ‘0-’ and ‘L-’.

In solutions, the open-chain form of glucose (either ‘0-’ or ‘L-’) exists in equilibrium with several cyclic isomers, each containing a ring of carbons closed by one oxygen atom. In aqueous solution, however, glucose exists as pyranose for more than 99%. The open-chain form is limited to about 0.25% and furanose exists in negligible amounts. The terms “glucose” and “D-glucose” are generally used for these cyclic forms as well. The ring arises from the open-chain form by a nucleophilic addition reaction between the aldehyde group —(C═O)H at C-1 and the hydroxyl group —OH at C-4 or C-5, yielding a hemiacetal group —C(OH)H—O—.

The reaction between C-1 and C-5 creates a molecule with a six-membered ring, called pyranose, after the cyclic ether pyran, the simplest molecule with the same carbon-oxygen ring. The (much rarer) reaction between C-1 and C-4 creates a molecule with a five-membered ring, called furanose, after the cyclic ether furan. In either case, each carbon in the ring has one hydrogen and one hydroxyl attached, except for the last carbon (C-4 or C-5) where the hydroxyl is replaced by the remainder of the open molecule (which is —(CHOH)2-H or —(CHOH)—H, respectively).

The ring-closing reaction makes carbon C-1 chiral, too, since its four bonds lead to —H, to —OH, to carbon C-2, and to the ring oxygen. These four parts of the molecule may be arranged around C-1 (the anomeric carbon) in two distinct ways, deSignated by the prefixes ‘a-’ and ‘p-’. When a glucopyranose molecule is drawn in the Haworth projection, the deSignation ‘a-’ means that the hydroxyl group attached to C-1 and the —CH20H group at C-5 lies on opposite sides of the ring's plane (a trans arrangement), while ‘p-’ means that they are on the same side of the plane (a cis arrangement).

Therefore, the open isomer D-glucose gives rise to four distinct cyclic isomers: a-D-glucopyranose, P-D-glucopyranose, a-D-glucofuranose, and P-Dglucofuranose; which are all chiral.

Polymers of glucose or dextrose are also useful such as polydextrose, starch and/or amylase. Polysaccharides generally have the formula of Cx(H20)y or may take the formula of (C6H100 5)17 with” ranging from 40 to 1 000. Polysaccharides include starches, glycogen, cellulose and chitin.

### Buffer

The presence of some buffering anion, e.g., an anion selected from acetate and/or lactate, in the dialysate precursor composition allows the dialysate precursor composition to be used as the acid concentrate in a standard three-stream dialysis machine, along with standard base (Le., bicarbonate) concentrate, thereby mitigating problems associated with fluctuations in the pH of the dialysate during a dialysis treatment. Absent the buffering anion, the dialysate can have pH and/or conductivity properties which are outside the ranges considered acceptable by health care professionals. The composition used in the method of the invention has a pH ranging from about 6.9 to 7.4, preferably from about 7.0 to 7.4, and more preferably from about 7.2 to 7.3.

Generally, a buffer is added to the composition of the invention. Salts of carbonates, lactates, citrates, and acetates are also useful as buffers in their salt form. Useful citrates include citric acid, sodium dihydrogen citrate, disodium hydrogen citrate, trisodium citrate, trisodium citrate dihydrate, potassium dihydrogen citrate, and dipotassium hydrogen citrate among others.

Useful acetates include acetic acid, sodium acetate, sodium acetate trihydrate, and potassium acetate, among others. Lactates useful in the composition of the invention include lactic acid, sodium lactate, potassium lactate, any variety of lactate dihydrates or trihydrates.

By far the more preferred buffers are carbonate and bicarbonate buffering to a pH ranging from about 7.2 to 7.3. The body provides a self balancing system for pH control. Respiration, urination, and other bodily functions assist in balancing pH. Certain buffers assist in modulating pH but are acidic and before neutralization can burn body or peritoneal tissue. Other buffers if built up in the body can be toxic at certain concentrations. Still further, some buffers are difficult to metabolize often presenting a complex pathway, metabolized through the liver. Here again, the ultimate metabolite may function effectively as a buffer but can present toxicity in certain concentrations.

Bicarbonate and salts of the carbonate ion are preferred. The bicarbonate ion (hydrogen carbonate ion) is an anion with the empirical formula HC0₃- and a molecular mass of 61.01 daltons; it consists of one central carbon atom surrounded by three oxygen atoms in a trigonal planar arrangement, with a hydrogen atom attached to one of the oxygens. It is isoelectronic with nitric acid HN03. The bicarbonate ion carries a negative one formal charge and is the conjugate base of carbonic acid H₂C0₃; it is the conjugate acid of CO₃²⁻, the carbonate ion as shown by these equilibrium reactions.

CO₃²⁻+2 H₂0-HC0₃⁻+H₂0+OH⁻—H₂C0₃+2 OH⁻

H₂C0₃+2 H20-HC0₃⁻+H₃0⁺+H₂0-CO₃²⁻+2 H30⁺

A bicarbonate salt forms when a positively charged ion attaches to the negatively charged oxygen atoms of the ion, forming an ionic compound. Many bicarbonates are soluble in water at standard temperature and pressure, in particular sodium bicarbonate and magnesium bicarbonate; both of these substances contribute to total dissolved solids, a common parameter for assessing water quality.

Bicarbonate is alkaline, and a vital component of the pH buffering system of the human body (maintaining acid-base homeostasis). 70 to 75 percent of CO2 in the body is converted into carbonic acid (H2C03), which can quickly turn into bicarbonate (HC03-).

With carbonic acid as the central intermediate species, bicarbonate—in conjunction with water, hydrogen ions, and carbon dioxide—forms this buffering system, which is maintained at the volatile equilibrium required to provide prompt resistance to drastic pH changes in both the acidic and basic directions. This is especially important for protecting tissues of the central nervous system, where pH changes too far outside of the normal range in either direction could prove disastrous.

Bicarbonate also acts to regulate pH in the small intestine. It is released from the pancreas in response to the hormone secretin to neutralize the acidic chyme entering the duodenum from the stomach.

### Ions

Generally, the composition of the invention comprises constituents which maintain the conductivity across cell membranes. Cations such as sodium and potassium are examples of ions which accomplish this function. Additionally, these cations work in concert with the sugars in regulating water content within the body. Any number of constituents may be used consistent with these functions.

As used herein, the phrase “physiologically acceptable cations” refers to cations normally found in the blood, plasma, or serum of a mammal, or cations that may be tolerated when introduced into a mammal. Suitable cations include protons, ammonium cations and metal cations. Suitable metal cations include, but are not limited to, the cationic forms of sodium and potassium, where sodium and potassium are preferred, and sodium is more preferred.

An ammonium cation, i.e., a compound of the formula —N+ where R is hydrogen or an organic group, and may be used so long as it is physiologically acceptable. In a preferred embodiment, the cation is selected from hydrogen (i.e., proton), sodium, potassium, among others. In its most preferred form the invention is free of calcium, magnesium, and combinations thereof.

Also useful in the composition of the invention is chloride ion. The chloride ion assists in balancing pH and osmolality of the solution used in the method of the invention. Generally osmolality in the human body is about 30 miliosmoles per deciliter. Chloride is generally introduced into the solution in salt from (Na+Cr) through any physiologically acceptable salt. Plasma osmolality is determined by the concentrations of the different solutes in the plasma. Solutes such as physiologically acceptable sodium salts (chloride and bicarbonate), glucose and urea are all the primary contributing salts. One estimate of plasma osmolality is defined by:

Calculated Plasma Osmolality=(2×plasma [Na))+[glucose]+[urea]

Using standard units.

### Diluent

A preferred water of the invention is treated in order that it is essentially pyrogen-free and sterile, and at least meets the purity requirements established by the Association for the Advancement of Medical Instrumentation (AAMI) for dialysate compositions. The water may also be referred to as treated water or AAMI-quality water. A monograph describing water treatment for dialysate, monitoring of water treatment systems, and regulation of water treatment systems is available from AAMI (Standards Collection, Volume 3, Dialysis, Section 3.2 Water Quality for Dialysis, 3 ed., 1998, AAMI, 3330 Washington Boulevard, Arlington Va. 22201) or through the Internet at http://www.aami.com. In addition, all of the other components of the precursor dialysate composition of the present invention are preferably at least United States Pharmacopeia (USP)-grade purity, which is generally a purity of about 95%. The purity of the components is preferably at least about 95%, more preferably at least about 98%, and more preferably at least about 99%.

The treated water may be obtained by following standard purification techniques, including, for example, distillation and reverse osmosis. Alternatively, the treated water may be purchased commercially. Such treated water is used in all, or nearly all, dialysis clinics and, accordingly, is well known to one of ordinary skill in the art.

As anyone skilled in the art will know the dialysate solution of the invention may comprise any number of constituents at varying concentrations depending upon the individual needs of the patient. In the home setting, the patient may be capable of optimizing constituent concentration. The composition of the invention may be prepared from dry form or administered in liquid form in a rigid or flexible container. Useful containers have been found to be 0.5 liters to 10 liters.

Useful concentrations are found below in the Table.

TABLE
(m/Eg/l)

| | USEFUL | PREFERRED | MORE PREFERRED |
|---|---|---|---|
| SUGAR* | 12-70 | 15-60 | 15-42.5 |
| BUFFER (NaHCO₃ | 32-42 | 35-40 | 36-38 |
| SODIUM | 120-138 | 126-130 | 126-130 |
| CHLORIDE | 94-102 | 94-100 | 96-98 |
| WATER | q.s | q.s | q.s |

*gm/L

### Method of Use

One of the advantages of peritoneal dialysis is its continuous nature. There are essentially three steps in peritoneal dialysis; fill, dwell and drain. In filling, the patients peritoneal cavity is filled with the sterile fresh dialysis solution. The patient then leaves the fluid in the peritoneal cavity for a set period of time. After this dwelling period, the patient serves the used diasylate from the peritoneal cavity and it is drained into the collection bag.

In use, the peritoneal membrane serves as a diffusion membrane between the peritoneal cavity and the blood. Solutes such as urea and creatinine move from areas of higher concentration to lower concentration. Water also moves across the membrane according to osmolar gradient.

Those factors which affect diffusion include the size of the peritoneal membrane. Scaring or surgery which alters the size or permeability of the membrane may limit the rate of diffusion.

Solute characteristics also alter the rate of diffusion. Equilibration across the membrane may be affected by solute size and molecular weight, molecular charge, protein binding capacity and the time and temperature of dwell among other factors. The concentration gradient will also affect dwell including the diffusion rate. Finally, among a great number of factors, capillary blood flow will affect diffusion in that the more optimal bloodflow is, the better the exchange rate in diffusion.

Similarly, ultrafiltration relies on many of the same indicia including surface area and permeability of the peritoneal membrane along with the overall health of the peritoneal membrane. Also relevant are dwell time, hydrostatic pressure gradient, colloid osmotic pressure and lymphatic reabsorption.

### WORKING EXAMPLE

The following example provides a nonlimiting illustration of one embodiment of the invention.

Although peritoneal dialysis (“PD”) has been effectively used for more than three decades to treat patients with irreversible Renal Failure, the number of Patients treated with this modality has decreased recently for many reasons.

It is recognized that the continuous nature of PD provides better control of symptoms. Elimination of calcium from the blood during Hemodialysis has been tried with disastrous results because acute Hypocalcemia ensues and causes tetanny bleeding, due to calcium. Calcium is essential for normal coagulation but may cause cardiac arrest in systole. On the other hand, PD presents itself as the ideal setting for the removal of calcium from the dialysate. Being that an average of 10 Liters of equilibrated (spent) dialysate are removed from a patient in 24 hrs., a small negative calcium balance is achieved. A patient on calcium-free peritoneal dialysis would lose less calcium through dialysis than do normal individuals through urine. This may prove to be beneficial to the cardiovascular system.

Hyperphosphatemia is a common result of renal failure that leads to bone disease in patients. The use of calcium-based phosphate binders is being abandoned in favor of more expensive, less effective salts such as lanthanum. Using calcium/magnesium-free dialysate would allow the return to this effective and costeffective practice. Additionally, a dialysate formulation that includes bicarbonate as the buffer, eliminating the precipitation of calcium/magnesium carbonate, in a solution containing a neutral pH, is preferred. Current dialysate solutions use lactate buffers which are converted in the body into bicarbonates. Before this conversion, the acidic nature of the lactates irritates the peritoneal tissue.

Normocalcemic patients using calcium- and magnesium-free dialysis would lose less calcium/magnesium through dialysis than do normal individuals through urine. The magnesium in the dialysate is already reduced relative to the normal plasma levels and thus is replaced orally.

A calcium carbonate/magnesium sulfate in 90/10% ratio is administered orally as a phosphate binder with meals or as a food supplement between them. The use of a calcium-free bicarbonate solution for PO has multiple benefits. It provides the physiologic buffer thus eliminating the deleterious effect of lactate (acidic) containing solutions on the peritoneal membrane. This solution prevents calcium deposition in soft tissue resulting from the absorption of calcium from the dialysate and reduces the risks of cardiovascular morbidity/mortality.

This solution also allows better phosphate control with Calcium salts which are not only effective but of low cost and more palatable. Vitamin D analogues could be administered without risk of hypercalcemia and slowing bone remodeling.

This physiologic Dialysis Solution, in concert with additional technological improvements, improves patient/technique survival and leads to lower costs of health care delivery to patients suffering from irreversible renal failure. This a very important issue considering that the number of patients in need of dialysis therapy is to increase tremendously in coming years.

The formulation and manufacture of a bicarbonate containing PD Solution devoid of Calcium, Magnesium or any other precipitable salts and an adjusted pH to 7.5 to 8.0 was completed.

The manufacture of a bicarbonate containing PD Solution packaged in Glass; Rigid Plastic Container or flexible plastic bags in volumes of 250 to 5000 ml

### Typical Formulation for 100 ml Application

| | |
|---|---|
| Dextrose hydrous | 1.5; 2.5, 4.25, 6.0 or 7.0% |
| Sodium bicarbonate | 448 mg |
| Sodium chloride | 538 mg |
| Water for injection | qs |

Approximate milliequivalents per liter: Sodium 132; Bicarbonate 40; Chloride 95

Treatment of soft tissue calcification in hypercalemic states is largely preventive and rarely attenuates existing extraosseous calcium deposition. Hemodialysis using low Calcium or Calcium-free baths abruptly lowers serum Calcium, and hence potentiates hypocalcemia and its attendant cardiac and neuromuscular complications. Continuous ambulatory peritoneal dialysis (CAPD), with its limited capacity for solute removal, prevents Calcium efflux beyond plasma/dialysate equilibrium.

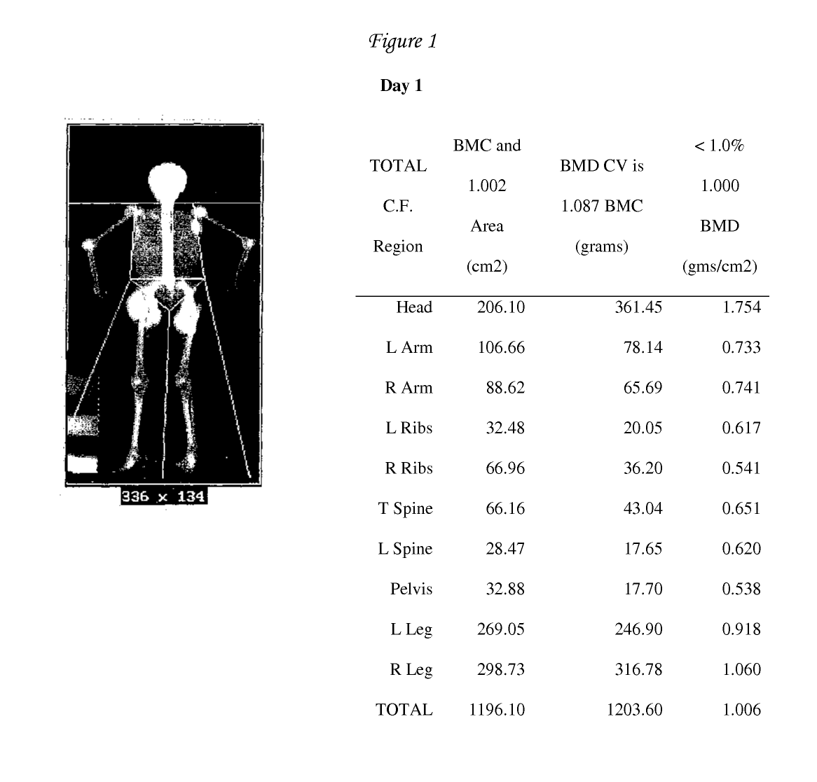

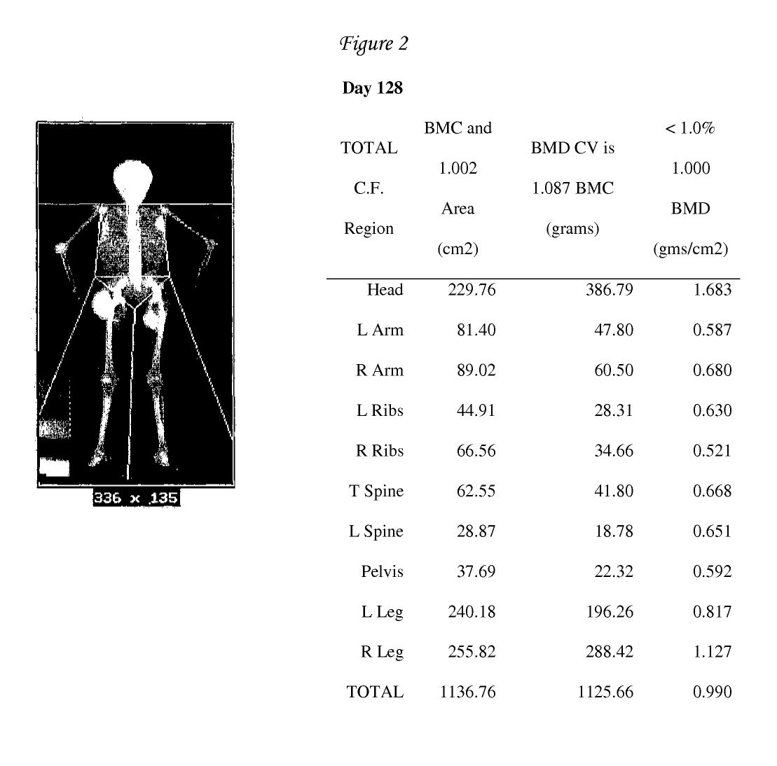

A 28 year-old female with juvenile rheumatoid arthritis, idiopathic recalcitrant hypercalcemia with extensive soft tissue calcifications and renal failure secondary to nephrocalcinosis and nephrolithiasis was treated for over 500 days with calcium-free CAPD. Hypercalcemia had not normalized on prednisone (80 mg daily), with corrected CA remaining 12-14 mg/dl. Intact parathyroid hormone levels (PTH) were undetectable; 25-hydroxyvitamin D, alkaline phosphatase were normal and serum phosphorous moderately elevated. Radiographs showed massive periarticular Calcium deposits on hips, shoulders, elbows and hands, FIGS. 1-3.

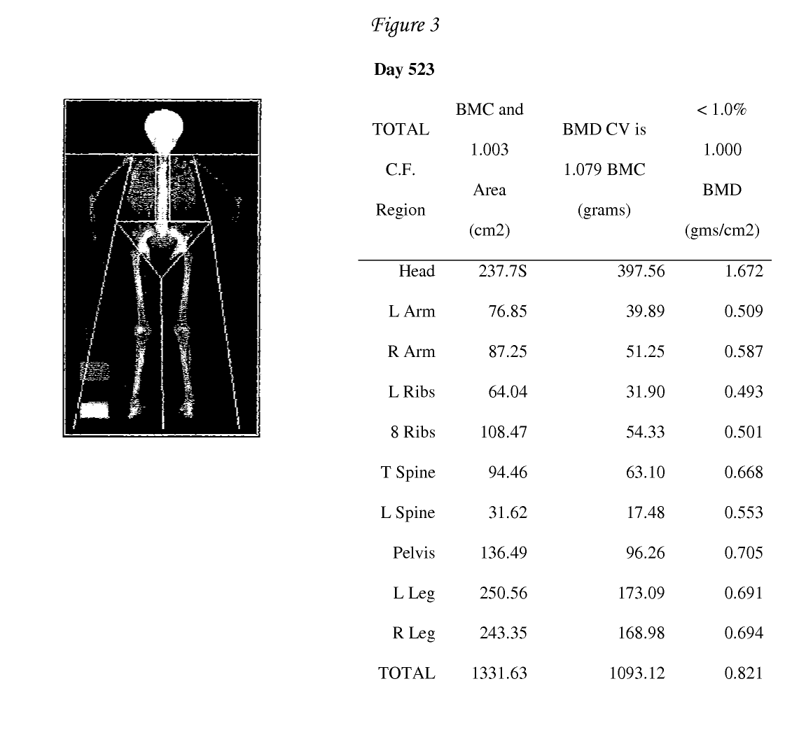

Removal of 4.0 to 5.6 mg of Calcium per dl of dialysis effluent lowered CCA to 10.4 mg/dl early on, despite immediate reduction of prednisone dose to 10 mg on alternate days. Daily calcium removal averaging 289 mg over 7 months has maintained normocalcemia; low-dose prednisone has been needed for arthritic complaints. PTH levels have gone from nil to low normal levels. Symptomatic and functional improvement has parallelled physical and radiographic evidence of a reduction in size of deposits. Serial dual energy X-ray absorptometry studies confirm a decrease in size and density of calcium deposition at elbows, shoulders, hips and about hands. Calcium free CAPO offers the promise of a safe and effective management of chronic hypercalcemia and reduces extraosseous calcium deposition without apparent bone demineralization, FIG. 3.

While the invention has been described above according to its preferred embodiments of the present invention and examples of steps and elements thereof, it may be modified within the spirit and scope of this disclosure. This application is therefore intended to cover any variations, uses, or adaptations of the instant invention using the general principles disclosed herein. Further, this application is intended to cover such departures from the present disclosure as come within the known or customary practice in the art to which this invention pertains and which fall within the limits of the following claims.

1. A method of delivering a solution to a kidney dialysis patient, said method comprising the steps of:
a. infusing a solution comprising:
(i) an amount of sugar effective to create an osmotic effect;
(ii) an effective amount of buffering agent;
(iii) sodium;
(iv) chloride;
(v) a balance of water; and
b. withdrawing the solution from the dialysis patient's peritoneum.

2. The method of claim 1, wherein said dialysis solution is free of calcium.

3. The method of claim 1, wherein said dialysis solution is free of magnesium.

4. The method of claim 7, wherein said sugar comprises a dextrose polymer.

5. The method of claim 1, wherein said sugar is selected from the group consisting of dextrose, starch, polysaccharide, glycogen, and mixtures thereof.

6. The method of claim 1, wherein said sugars comprise a dextrose polymer present in a concentration ranging from about 1.5% to 6%.

7. The method of claim 1, wherein said buffering agent comprises a carbonate salt.

8. The method of claim 1, wherein said buffering agent is selected from the group consisting of a citrate, a lactate, a carbonate, and mixtures thereof.

9. The method of claim 1, wherein said buffering agent comprises sodium bicarbonate and provides a pH ranging from about 7.3 to 7.4.

10. The method of claim 1, wherein sodium is present in a concentration ranging from about 120 to 138 gmtl.
of:

11. A method of delivering an aqueous dialysis solution comprising the steps
a. withdrawing fluid from the peritoneum; and
b. infusing said aqueous dialysis solution into said peritoneum, said aqueous solution comprising a dextrose polymer, a buffering agent, and sodium chloride;
wherein said solution has a pH ranging from about 7.3 to 7.4, a sodium concentration of about 126 to 130 gm/l, and a chloride concentration of about 94 to 100 gm/l.

12. The method of claim 11, wherein said dialysis solution is free of calcium.

13. The method of claim 11, wherein said dialysis solution is free of magnesium.

14. The method of claim 11, wherein said sugar is selected from the group consisting of dextrose, starch, polysaccharide, glycogen, and mixtures thereof.

15. The method of claim 11, wherein said sugars comprise a dextrose polymer present in a concentration ranging from about 1.5 to 7.

16. The method of claim 11, wherein said buffering agent comprises a carbonate salt.

17. The method of claim 11, wherein said buffering agent is selected from the group consisting of a citrate, a lactate, a carbonate, and mixtures thereof.

18. The method of claim 11, wherein said buffering agent comprises sodium bicarbonate and provides a pH ranging from about 7.3 to 7.4.

19. The method of claim 11, wherein chloride is present in a concentration ranging from about 96 to 98.

20. A peritoneal dialysis solution comprising:
a. from about 120 to 138 gm/l of sodium and about 12 to 60 gm/l of chloride;
b. an amount of dextrose polymer ranging from about 12 to 70 gm/l;
c. a carbonate buffering agent present in an amount necessary to provide a pH ranging from about 7.3 to 7.4; and
d. a balance of water wherein said solution is free of calcium and free of magnesium.
